(19) United States
(12) Patent Application Publication
(10) Pub. No.: US 2025/0067173 A1
(43) Pub. Date: Feb. 27, 2025

(54) BENCHTOP AUTOMATED CUTTINGS IMAGING AND ANALYSIS

(71) Applicant: Halliburton Energy Services, Inc., Houston, TX (US)

(72) Inventors: Mathew Dennis Rowe, Houston, TX (US); Shaun Patrick Lawrence, Houston, TX (US)

(21) Appl. No.: 18/455,319

(22) Filed: Aug. 24, 2023

(51) Int. Cl. E21B 49/00; E21B 44/00

(52) U.S. Cl. CPC E21B 49/005; G01N 35/00029; G01N 33/24; G01N 2035/00039

(57) ABSTRACT

Some implementations include a method for analyzing cuttings from a plurality of depths while drilling a wellbore in a subsurface formation, the method comprising: obtaining cuttings samples from the plurality of depths while drilling the wellbore in the subsurface formation; performing the following operations for each of the cuttings samples: loading a cuttings sample into a viewing area of a microscope coupled to an image capture device and a computer having a learning machine, performing analyses on the cuttings sample, and capturing, via the image capture device, a plurality of images of the cuttings sample through the microscope. The method further includes outputting a standardized cuttings report generated by the learning machine.

### FIELD

Some implementations relate generally to the field of obtaining measurements of drill cuttings and, more particularly, to the field of automated sampling of drill cuttings using chemicals and multiple light spectra.

### BACKGROUND

In drilling of a wellbore in a subsurface formation, cuttings are formed when a drill bit shaves or otherwise breaks away pieces of the formation to form the wellbore. The cuttings then return to surface via flow of a drilling fluid. Samples of the cuttings may be obtained for analysis to assist in characterizing the subsurface formation and perform drilling operations. The analysis may include obtaining cuttings sample measurements that may indicate the type of formation being drilled, changes in the subsurface formation properties with respect to depth, etc. Traditionally, an on-site geologist may perform the analysis on the cuttings and provide descriptions that may be subject to human bias—each person may describe the same cuttings sample differently.

### BRIEF DESCRIPTION OF THE DRAWINGS

Implementation of the disclosure may be better understood by referencing the accompanying drawings.

FIG. 1 is an illustration depicting an example well system, according to some implementations.

FIG. 2 is an illustration depicting an example automated cuttings analysis system, according to some implementations.

FIG. 3 is a flow diagram depicting a training procedure for an example learning machine, according to some implementations.

FIG. 4 is a flow diagram showing operations for predicting, by a trained learning machine, one or more properties of cuttings, according to some implementations.

FIG. 5 is a flowchart of example operations for analyzing a cuttings sample using the automated cuttings analysis system, according to some implementations.

FIG. 6 is a block diagram depicting an example computer, according to some implementations.

### DESCRIPTION

The description that follows includes example systems, methods, techniques, and program flows that embody aspects of the disclosure. However, it is understood that this disclosure may be practiced without these specific details. For instance, this disclosure refers to one or more automated devices configured to perform cuttings analysis under multiple spectra of light with the addition of chemicals to determine the mineralogy of the cuttings. Aspects of this disclosure may also be applied to any other configuration of devices configured to perform cuttings analysis. For clarity, some well-known instruction instances, protocols, structures, and techniques have been omitted.

Example implementations relate to automated cuttings analysis while drilling a wellbore in a subsurface formation. In some implementations, devices configured to obtain measurements of cuttings samples may be utilized to glean subsurface formation properties such as chemical composition of the rock, changes in lithological properties, changes in provenance, changes in diagenetic environment, mineralogy, etc. The devices may be used as part of a system configured to obtain other cuttings properties including, but not limited to cutting size, shape, color, roundness, angularity, feret diameter, grain size, percent dolomite composition, percent limestone composition, caving(s) volume, silt/clay content (as determined via a methylene blue test), etc. In some implementations, the cuttings may be prepared in a certain state (such as dried, crushed, cleaned, etc.) to properly obtain measurements of the cuttings samples.

In some implementations, an automated cuttings analysis system including a microscope, one or more lights, and one or more fluid storage vessels may be configured to perform analyses on cuttings samples obtained from a wellbore being drilled. Cuttings samples from a plurality of depths may be obtained from the wellbore during the drilling. Each cuttings sample may be loaded into a standardized tray or similar container, and container may be loaded into an autoloader. Some implementations may load cuttings samples into cartridges. The autoloader may be configured to load and unload each cartridge from a viewing area under the microscope. The autoloader may be connected to the microscope, where the microscope may be configured to operate in visible light, UV light, or other light spectrums. The microscope may include a charge-coupled device (CCD) camera, a hyperspectral detector, or other image capture device(s). The microscope may also have one or more automated dosing devices (autodosers) for distributing hydrochloric (HCl) acid, phenolphthalein, and/or other liquid chemicals on to the cuttings sample to determine the cutting sample's reactivity to the one or more chemicals. The microscope may be controlled by a computer or other controller system. When cuttings sample is loaded into the viewing area of the microscope, the automated cuttings analysis system may be configured to capture images of the sample in multiple light spectra, dose the cuttings with various chemicals, and record the results of the dosing (via the image capture device). The images may be transferred to the computer, and the computer may include one or more machine learning (ML) models and/or artificial intelligence (AI) to determine the mineralogy of the cuttings based on the images. The ML model(s) or AI may output results using standard cuttings descriptors. These results may be output to a collaborative database and may be organized according to a depth at which a cuttings sample was obtained. Results may be standardized across all cuttings, reducing biases introduced via cuttings examination by geologists. In some implementations, captured images may be labeled with the depth at which the cuttings sample in view was obtained, and the results may be reported.

In some implementations, subsurface operations may be performed based on the results output by the automated cuttings analysis device. For example, a well log formed by sequencing cuttings properties at their respective depths. This well log may be compared to offset well data and/or core samples to correlate subsurface formations while drilling. Accordingly, subsurface operations may be performed based on properties of the cuttings. For instance, drilling parameters (e.g., weight-on-bit, torque-on-bit, mud weight, etc.) may be adjusted, the drill bit and/or other drilling components on the drilling assembly may be replaced, etc.

### Example Systems

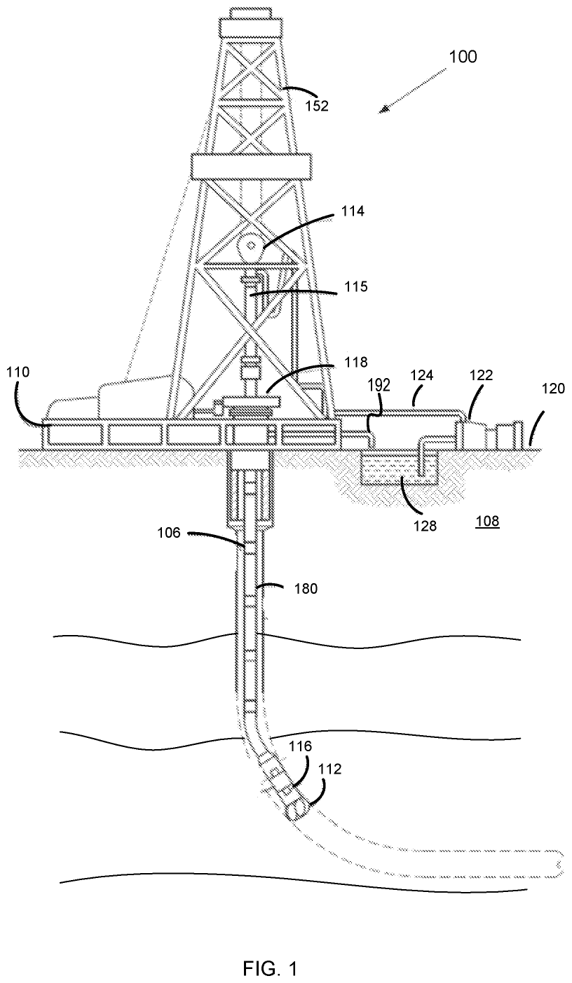

FIG. 1 is an illustration depicting an example well system, according to some implementations. In particular, FIG. 1 is a schematic diagram of a well system 100 that includes a drill string 180 having a drill bit 112 disposed in a wellbore 106 for drilling the wellbore 106 in the subsurface formation 108. While depicted for a land-based well system, example implementations may be used in subsea operations that employ floating or sea-based platforms and rigs.

The well system 100 may further include a drilling platform 110 that supports a derrick 152 having a traveling block 114 for raising and lowering the drill string 180. The drill string 180 may include, but is not limited to, drill pipe, drill collars, and drilling assembly 116. The drilling assembly 116 may comprise any of a number of different types of tools including a rotary steerable system (RSS), measurement while drilling (MWD) tools, logging while drilling (LWD) tools, mud motors, etc. A kelly 115 may support the drill string 180 as it may be lowered through a rotary table 118. The drill bit 112 may include roller cone bits, polycrystalline diamond compact (PDC) bits, natural diamond bits, any hole openers, reamers, coring bits, and the like. Drilling parameters of drilling the wellbore 106 may be adjusted to increase, decrease, and/or maintain the rate of penetration (ROP) of the drill bit 112 through the subsurface formation 108 and, additionally, steer the drill bit 112 through the subsurface formation 108. Drilling parameters may include weight-on-bit (WOB), torque-on-bit (TOB), mud weight, and rotations-per-minute (RPM) of the drill string 180.

A pump 122 may circulate drilling fluid through a feed pipe 124 to the kelly 115, downhole through interior of the drill string 180, through orifices in the drill bit 112, back to the surface 120 via an annulus surrounding the drill string 180, and into a retention pit 128 via flowline 192. The drilling fluid may carry cuttings up the annulus to surface where they may be separated from the drilling fluid on surface before the drilling fluid flows into the retention pit 128. For example, the drilling fluid and cuttings may pass through a shale shaker (not pictured) to separate the cuttings from the drilling fluid and collect the cuttings in an apparatus. Samples of the cuttings may be obtained from the apparatus for further analysis to assist in analyzing the subsurface formation.

### Example Automated Cuttings Analysis System

Examples of an automated cuttings analysis system are now described.

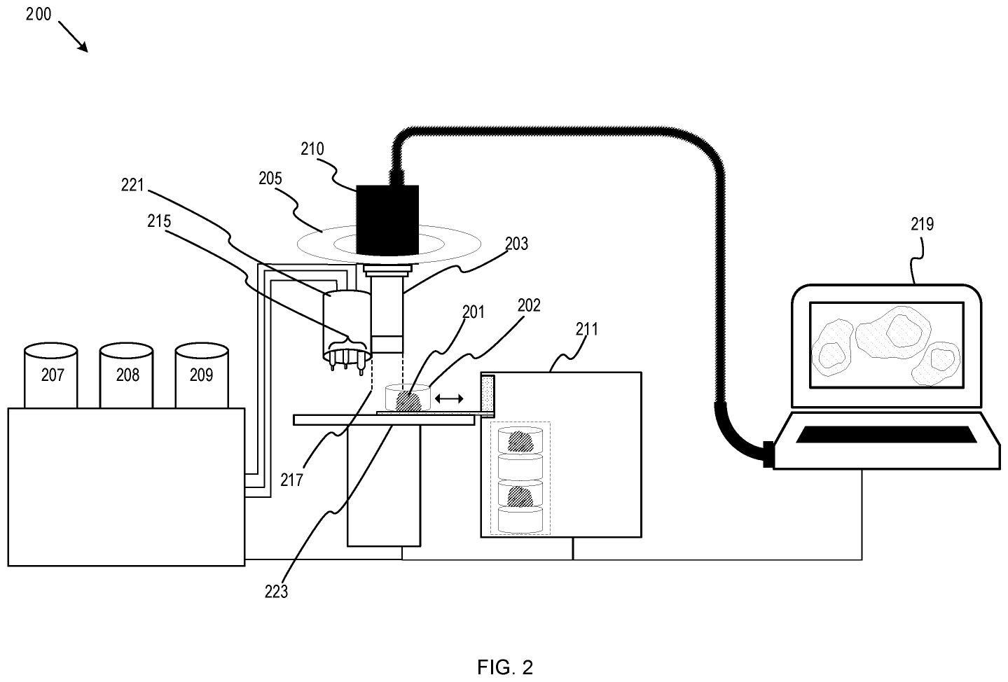

FIG. 2 is an illustration depicting an example automated cuttings analysis system, according to some implementations. The automated cuttings analysis system 200 may be located within an on-site laboratory proximate to the well system 100 of FIG. 1. The automated cuttings analysis system 200 may be configured to perform a benchtop automated analysis of a cuttings sample 201. The cuttings sample 201 (also referred to as the “sample 201”) may be retrieved from a shale shaker proximate to the well system 100. The cuttings sample 201 may include a single piece of drill cuttings, but other implementations may classify multiple cuttings fragments as the cuttings sample 101. The cuttings sample 201 may be brought to the laboratory by on-site personnel. In some implementations, the sample 201 may undergo preparation prior to analysis by the automated cuttings analysis system 200. The preparation may include washing the cuttings sample 201, drying the sample, etc. In some implementations, the sample 201 may be placed into an oven situated within the laboratory to dry, and the sample 201 may be centrifuged.

The automated cuttings analysis system 200 may include a microscope 203 having a viewing area 217, a light source 205, one or more fluid storage vessels 207-209, an image capture device 210, an autoloader 211, autodosers 215, and a computer 219. The automated cuttings analysis system 200 may be configured to analyze the cuttings sample 201. In some implementations, the cuttings sample 201 may be placed on or housed within a container 202. Some implementations of the container 202 may include a standardized tray, a microscope slide, a low-rise bowl, and a cartridge, although other configurations may be possible. Some implementations of the autoloader 211 may be configured to move the container 202 within the viewing area 217 of the microscope 203. In other implementations, the autoloader 211 may be configured to load the cuttings sample into the container 202.

Conventional approaches performed by on-site geologists may utilize light spectra and chemicals to describe cuttings samples. For example, a geologist may use ambient light (also referred to as standard or visible light) to look at cuttings and describe their color, shape, and other properties. Geologists may also use ultraviolet (UV) light to analyze the cuttings, as hydrocarbons and certain minerals such as calcite, fluorite, etc. fluoresce under UV light. However, the automated cuttings analysis system 200 may be configured for additional analyses. For example, the microscope 203 may be coupled to the image capture device 210, where the image capture device 210 may be configured to capture images of the sample 201 under multiple light spectra. Some implementations of the microscope 203 may utilize a stereo microscope, although other configurations may be possible. In some implementations, the microscope 203 may include a movable table 223 to assist in aligning the cuttings sample 201 with the viewing area 217, the autodosers 215, etc.

In some implementations, the image capture device 210 may include a CCD camera/sensor. The CCD sensor may be a transistorized light sensor etched onto an integrated circuit for detecting light spectra including, but not limited to visible light, UV light, near-infrared light, etc. In some implementations, the image capture device 210 may be a red, green, and blue (RBG) camera coupled with the CCD sensor. In other implementations, the image capture device 210 may be an RGB-D (RGB-depth) camera which uses the CCD sensor to map depth for three-dimensional (3D) imaging of the sample 201. In other implementations, the image capture device 210 may be a Three-CCD (3CCD) camera which uses a separate CCD sensor for each of a red, green, and blue color range. In some implementations, the image capture device 210 may be configured to capture a video of the cuttings sample 201. The video may record, for example, a reaction of the cuttings sample 201 with chemicals output from the autodosers 215.

As referenced above, the automated cuttings analysis system 200 may include a container 202. The container 202 may be standardized such that it may be compatible with the automated cuttings analysis system 200. The cuttings sample 201 may be loaded into the container 202. The container 202 comprising the cuttings sample 201 may be labeled through standard methodology. For example, the label may include attributes such as time, depth, drilling fluid properties (e.g., mud weight), a well identifier, etc. The automated cuttings analysis system 200 may be configured with the autoloader 211 such that the container 202, comprising the cuttings sample 201, may be automatically loaded into the viewing area 217 of the automated cuttings analysis system 200. The autoloader 211 may be configured to handle multiple cuttings samples and sequentially load each cuttings sample into the viewing area 217. The autoloader 211 may also automatically unload the container 202 once the measurements have been obtained and proceed to load the next cuttings sample into the viewing area 217 for analysis.

In some implementations, the autoloader 211 may include a conveyor system in which cuttings samples are moved to and from the viewing area 217 by conveyor belt. In other implementations, the autoloader 211 may include a cartridge system configured to load and unload one or more standardized cartridges comprising cuttings sample(s). In some implementations, the cartridges may be loaded with cuttings samples by on-site personnel and fed into the cartridge system of the automated cuttings analysis system 200. The autoloader 211 may be configured to convey each cartridge comprising a cuttings sample into and out of a viewing area 217 of the microscope 203. In some implementations, the autoloader 211 may be configured to handle multiple cartridges and sequentially load each cuttings sample into the viewing area 217. The autoloader 211 may also automatically unload each cartridge once the measurements have been obtained, and the autoloader 211 may proceed to load the next cuttings sample into the viewing area 217 for analysis. In some implementations, the autoloader 211 may include an internal movable shelf system to move cartridges. In other implementations, the autoloader 211 may include a spring loaded cartridge system. However, any suitable method to move cuttings samples up or down within the autoloader may be used. The autoloader 211 may include an automated arm or similar apparatus to convey cartridges from the autoloader 211 to the viewing area 217.

The light source 205 may comprise one or more bulbs or strips configured to output light at desired spectra. Some implementations may use multiple light sources 205. In some implementations, the light source 205 may be configured to shine light on the sample 201 at wavelengths within ambient (visible) light, UV light, infrared light, and near-infrared light spectra, although other light spectra may be utilized. While the light source 205 is depicted as a light ring, the light source 205 may be of any suitable shape or configuration. For example, the light source 205 may comprise one or more light bulbs to achieve the various light spectra.

Some implementations of the image capture device 210 may be capable of hyperspectral imaging and other forms of spectroscopy. Some implementations may use one or more camera systems or sensors capable of capturing images across a spectral range of approximately 300 nm to 1700 nm, although other types of cameras may also be used. For example, the image capture device 210 may be a hyperspectral imaging (HSI) camera, an infrared (IR) imaging device to produce images for IR mapping, a Fourier transformed infrared (FTIR) device for FTIR mapping, a Raman spectrometer coupled to a one or more lasers, etc. The cuttings sample 201 may be viewed through a lens of the microscope 203, images may be captured by the image capture device 210, and the images may be uploaded to the computer 219 for additional processing. In some implementations, the microscope 203 may be configured to achieve at least one of the above-described light spectra. Additional pictures may be taken at appropriate wavelengths, depending on the desired analyses.

The automated cuttings analysis system 200 may be configured to dose the cuttings sample 201 with chemicals during analysis. The microscope 203 may be coupled to the one or more fluid storage vessels 207-209. In some implementations, the one or more storage vessels 207-209 may be configured to store hydrochloric acid (HCl), phenolphthalein, isopropanol, etc., each in separate a vessel. Each storage vessel 207-209 may be coupled to its own autodoser of the autodosers 215. Each autodoser of the autodosers 215 may be configured to transport a chemical from its respective storage vessel 207-209 through separate conduits to avoid cross-contamination. In some implementations, fluid from the storage vessels 207-209 may be pumped through a pipe, a flexible hose, or a conduit to the autodosers 215. In some implementations, each autodoser of the autodosers 215 may be comprised of a dropper, a micro-doser, a pipette, a micro-pipette, or a micro peristaltic pump. In some implementations, the autodosers 215 may include a slanted or bent nose oriented towards the cuttings sample 201 for improved fluid delivery to the cuttings sample 201 without adjusting the movable table 223. However, other implementations and configurations for fluid dosing may be possible. Some implementations of the autodosers 215 may be included in a rotary assembly 221. The rotary assembly 221 be actuated with an electric motor or actuator to align a desired autodoser with the cuttings sample 201. In other implementations, the autodosers 215 may be positioned to dose the cuttings sample 201 without the rotary assembly 221.

One autodoser of the autodosers 215 may be coupled to a HCl storage vessel 207. This autodoser may dose the cuttings sample 201 with hydrochloric acid. Some implementations may use 10% HCl, although other concentrations may be used. The hydrochloric acid may fizz upon contact with any carbonates or dolomites within the cuttings sample 201. In some implementations, an autodoser of the autodosers 215 may be coupled to a phenolphthalein storage vessel 208 and configured to dose the sample with phenolphthalein. The phenolphthalein may be used to detect cement present in the cuttings sample 201, which may indicate cementing issues in the wellbore 106 of FIG. 1. The phenolphthalein may turn pink in the presence of cement. The presence of cement in the sample 201 may also signify where the cemented portion of the wellbore 106 of FIG. 1 ends and where a new formation begins.

In some implementations, an autodoser of the autodosers 215 may be coupled to a third fluid storage vessel 209. The third fluid storage vessel 209 may be configured to store isopropanol, butane, naphtha, or a similar solvent which may be used to expel hydrocarbons from the cuttings sample 201. In some implementations, the third fluid storage vessel 209 may be configured to store a surfactant that may be dosed on to the cuttings sample 201. In other implementations, the third fluid storage vessel 209 may be configured to store methylene blue. The methylene blue may be dosed onto the cuttings sample 201 to determine the presence and approximate amounts of certain clays in the cuttings sample 201, such as smectite. Once any liquid hydrocarbons within the cuttings sample 101 have been expelled, the light source 205 may shine a UV light on the fluid to determine its fluorescence. The color of the fluorescence may indicate a thermal maturity of the oil, with darker colors indicating heavier oils and lighter colors indicating lighter oils.

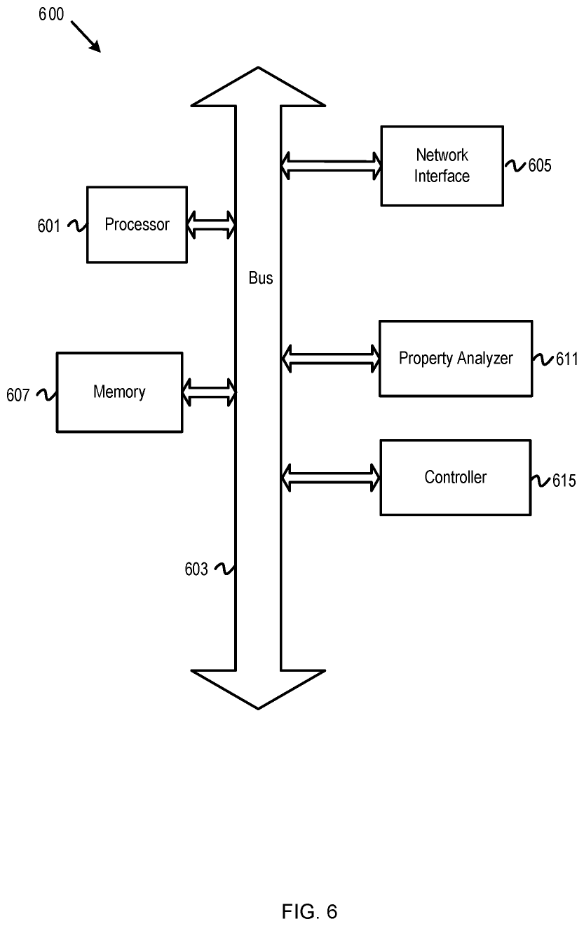

The automated cuttings analysis system 200 includes a computer 219 that may be communicatively coupled to its constituent components, such as the image capture device 210, fluid storage vessels 207-209, the autodosers 215, the light source 205, the autoloader 211, and the microscope 203. A processor of the computer 219 may perform simulations (as further described below). In some implementations, the processor of the computer 219 may be coupled to a controller configured to control cuttings analysis operations of the automated cuttings analysis system 200. For instance, the controller (later described as the controller 615) of the computer 219 may control the autoloader 211 to move the cuttings sample 201 or container 202, to dose the cuttings sample 201 via fluid output from the autodosers 215, to adjust lighting on the sample 201 via the light source 205, to capture images of the cuttings sample 201 via the image capture device 210, etc. An example of the computer 219 is depicted in FIG. 6, which is further described below.

### Example Learning Machine

The automated cuttings analysis system 200 may be configured to perform analyses as described above. A learning machine may be paired with the image capture device 210 and may be trained to identify features across various light spectra (UV, visible, IR, etc.). In some implementations, the learning machine may include a ML model. The ML model will be trained to identify different minerals based on images, whether they are hyperspectral, IR mapping, Raman mapping, FTIR, etc. The learning machine may also analyze other parameters besides mineralogy such as size, shape, color, roundness, angularity, feret diameter, grain size, etc. Images of one or more cuttings samples may be combined within the computer 219. The learning machine may analyze the images to determine one or more mineralogies of the overall composition, of various subsections of the wellbore, etc. The one or more mineralogies may also be referred to mineral contents, percent compositions, mineral composition, etc. Based on images, the learning machine may determine the percent compositions of minerals in the sample. The percent compositions, as well as standardized descriptors of the cuttings samples may then be output to a database that is accessible by geologists on and off the wellsite as well as other stakeholders to make decisions regarding a cross-correlation of wells, a true mineralogy of the wellbore, and decisions that alter the drilling operation (drilling ahead, to stop drilling, to set casing points, or determine if they are in a pay zone or not, etc.). In some implementations, the learning machine may be trained to determine a mineralogy based on standardized descriptors it has previously output.

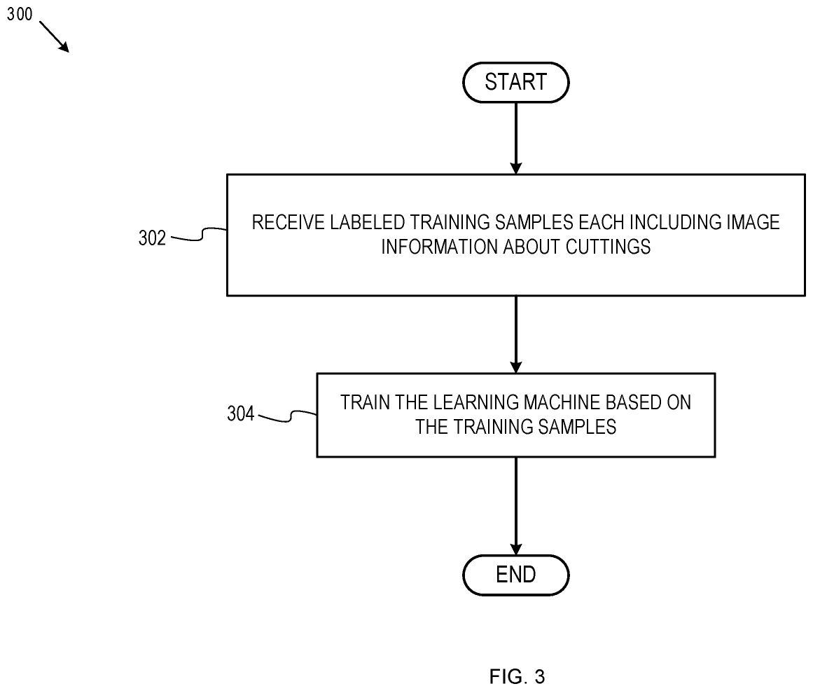

FIG. 3 is a flow diagram 300 depicting a training procedure for an example learning machine, according to some implementations. At block 302, a learning machine may receive labeled training samples, where each sample may include image information about cuttings from a borehole. Each sample may include any other information about the cuttings (such as depth). In some implementations, each training sample includes an image captured by the image capture device 210. Each image may depict one or more cuttings that were carried underneath the image capture device 210 by the autoloader 211. The learning machine may be configured to operate at wavelengths in the various light spectra used to analyze the cuttings sample 201. In the image, aspects of the cuttings may be labeled to indicate one or more ground truths such as minerals present in the cuttings. The learning machine may be trained to identify the minerals based on different images across various light spectra including hyperspectral images, infrared images, Raman images, FTIR images, images in visible light, etc.

At block 304, the learning machine may train itself to predict aspects of cuttings based on the labeled training samples received at block 302. For example, the learning machine may receive labeled images of cuttings. Based on the labeled images, the learning machine may perform training to predict minerals present (or other aspects) in the cuttings. The learning machine may include any suitable neural network (such as a convolutional neural network). In some implementations, the learning machine may process the training samples and perform feature extraction, back propagation, and other operations for training the neural network. The learning machine may use fewer than all the training samples in its training process. For example, the learning machine may utilize 80% of the training samples at block 304. Later, the learning machine may use the remaining 20% of the training samples to test itself.

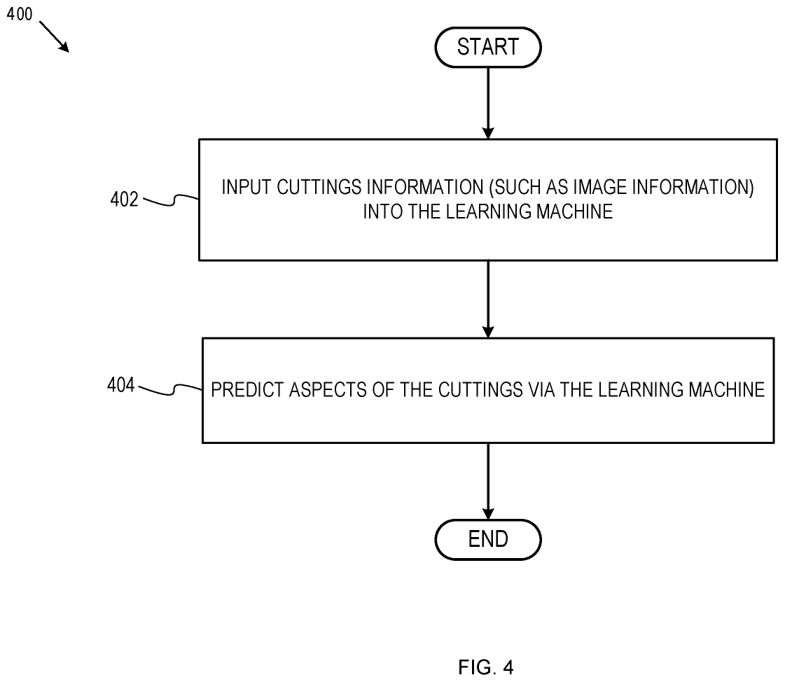

FIG. 4 is a flow diagram 400 showing operations for predicting, by a trained learning machine, one or more properties of cuttings, according to some implementations. At block 402, the learning machine receives information about cuttings. For example, the learning machine may receive an image of cuttings captured by the image capture device 210 and other information about the cuttings (such as a depth at which the cuttings were made).

At block 404, the learning machine predicts aspects of the cuttings. For example, the learning machine may identify one or more minerals present the cuttings sample based on the images. The learning machine also may identify or predict other aspects of the cuttings as noted above. For example, the learning machine may determine a percent composition of various minerals in the cuttings sample. The learning machine may also determine lithological, petrological, and other properties of the cuttings sample based on analyses, the properties including, but not limited to the depth at which the sample was obtained, color, estimated dolomite percent composition, estimated limestone percent composition, overall carbonate content, silt/clay content, fracture presence, consolidation, texture, swelling properties, macro structures, fluorescence and fluorescence color, cut, cut color, staining, rock type, caving volume, methylene blue test score, shape, angularity, roundness, feret diameter, grain size, grain shape, sorting, luster, rock matrix, estimated porosity, hydrocarbon show, etc. Some implementations of the learning machine may be configured to output general descriptors of the identified properties. Some implementations of the learning machine may also be configured to output general descriptors associated with the hydrocarbon show and estimated porosity. For example, the learning machine may determine a compensated oil show indicating the type of oil present in the cuttings sample. The learning machine may output a descriptor to describe the oil show (e.g., light oil, heavy oil, condensate oil, etc.). The learning machine may also output a compensated porosity descriptor based on the estimated porosity (e.g., good, poor, high/low streaming potential, etc.).

In some implementations, the learning machine generates a standardized cuttings report and stores the standardized cuttings report in a database. The standardized cuttings report may describe the minerology of the cuttings using standardized terminology. The standardized cuttings report may include any suitable information about the minerology or other aspects of the cuttings (such as depth, color, carbonate content, etc.). In some implementations, the learning machine may form short descriptions of cuttings samples based on a presence of multiple properties. For example, a cuttings sample with a notable silt/clay content and lack of fluorescence may be output as “Clay—12,500 ft MD” to denote its predominant lithology and the measured depth (MD) at which the sample was obtained. In other implementations, the learning machine may output multiple of the above listed properties.

In some implementations, output from the learning machine (such as a prediction about aspects of the cuttings) may be presented on any suitable output device or otherwise electronically transmitted to other computing devices for further analysis and/or presentation. In some implementations, the learning machine may output percent mineralogies, cuttings properties, and the depth at which they were obtained to a geological database accessible by operators, geologists, and other parties. Operators may undertake or modify drilling and/or surface operations based on predictions from the learning machine and from the standardized cuttings report.

### Example Operations

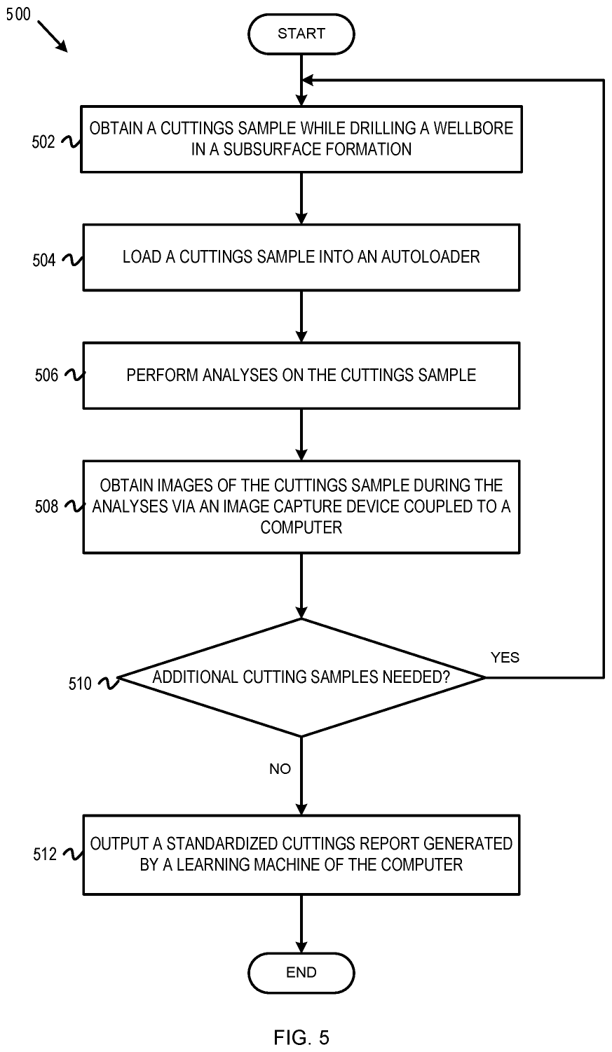

FIG. 5 is a flowchart of example operations for analyzing a cuttings sample using the automated cuttings analysis system, according to some implementations. FIG. 5 depicts a flowchart 500 of operations to obtain and measure properties of a cuttings sample under a microscope. The operations of flowchart 500 are described in reference to FIGS. 2-4. Additionally, a learning machine described in the operations of the flowchart 500 is described with reference to the computer 219 of FIG. 2 and with reference to FIGS. 3-4. Operations of the flowchart 500 begin at block 502.

At block 502, a cuttings sample 201 may be obtained while drilling a wellbore in a subsurface formation. The cuttings sample 201 may originate from a depth in the wellbore 106 during the drilling process, as described in FIG. 1. The cuttings sample 201 may be obtained from equipment on the drilling rig, such as the shale shaker. In some implementations, the cuttings sample may be obtained during other oil and gas operations, such as flowback operations. In some implementations, the cuttings sample 201 may be manually obtained by a sample catcher from a cuttings board representative of a range of depths in the wellbore of which the cuttings sample 201 was obtained. In other implementations, the cuttings sample 201 may be automatically obtained.

The cuttings sample 201 may be brought to the environment housing the automated cuttings analysis system 200. In some implementations, the cuttings sample 201 may be prepared via washing, drying, centrifuging, spinning, etc. In other implementations, the cuttings sample 201 may be unwashed cuttings (i.e., the cuttings do not have to be washed, dried, etc. before analysis). For example, the cuttings sample 201 may include cuttings as well as drilling fluids, lost circulation material, etc. The cuttings sample 201 may also be placed into a container 202. Flow progresses to block 504.

At block 504, cuttings sample 201 may be loaded into the autoloader 211. In some implementations, the container 202 comprising the cuttings sample 201 may be loaded into the autoloader 211. In some implementations, the container 202 may be loaded into the autoloader 211 by personnel such as the above-mentioned sample catcher. The capacity of the container may be completely full of a cuttings sample, partially full of a cuttings sample, etc. The autoloader 211 may utilize a conveyer belt system, a cartridge system, etc. to move the cuttings sample 201 into a viewing area of the microscope 203. Flow progresses to block 506.

At block 506, the automated cuttings analysis system 200 may perform analyses on the cuttings sample 201. For example, the automated cuttings analysis system may dose the cuttings sample 201 with one or more chemicals to test for cement presence, hydrocarbon presence, dolomite/carbonate presence, etc. The automated cuttings analysis system 200 may also illuminate the cuttings sample 201 via one or more light sources 205 in a plurality of light spectra at desired wavelengths for analysis. Flow progresses to block 508.

At block 508, the image capture device 210, coupled to the computer 219, may obtain images of the cuttings sample 201 during the analyses. For example, the image capture device 210 may capture images of the cuttings sample 201 viewed through the microscope 203. In some implementations, the image capture device 210 may capture images of one or more cuttings comprising the cuttings sample 201. Images of the cuttings sample 201 may be uploaded to the computer 219 and analyzed by a trained learning machine.

In some implementations, the computer 219 may label or otherwise associate images of each cuttings sample with the corresponding depth from which the cuttings sample originated. For example, if the cuttings sample originated from approximately 10,000 feet true vertical depth (TVD), then captured images of this sample may be labeled with the corresponding depth of 10,000 feet TVD. In some implementations, the images may be labeled with the corresponding measured depth (MD) of the cutting sample. In some implementations, the computer 219 may combine images of cuttings samples at their respective depths within a database to create a representation of the wellbore of which the cuttings were obtained. In some implementations, the trained learning machine may analyze multiple images at different depths to characterize a mineralogy of one or more depth intervals of the wellbore 106.

In some implementations, once the cuttings sample analyses have been performed and one or more images captured, the cuttings sample may be unloaded from the viewing area 217 to prepare for the next cuttings sample. For example, the autoloader 211 may unload the container 202, comprising the cuttings sample 201, from the viewing area 217 of the automated cuttings analysis system 200.

At block 510, a processor of the computer 219 may determine if additional cuttings samples are needed. If more cuttings sample are needed, then operations return to block 502 to obtain another cuttings sample for measuring. In some implementations, the next cutting sample may be from a similar or different depth than the depth of the prior cutting sample. If no additional cuttings samples are needed, then operations of the flowchart 500 proceed to block 512.

At block 512, a standardized cuttings report may be generated by a learning machine of the computer 219. For example, the learning machine may be trained to identify one or more minerals, one or more lithological properties, one or more petrological properties, etc. of the cuttings sample 201 based on images captured by the image capture device 210. The learning machine also may identify or predict other aspects of the cuttings as noted above. Based on the identified properties, the learning machine may output a standardized cuttings report including one or more standardized cuttings descriptors based on analyses performed on the cuttings sample 201. In some implementations, the standardized cuttings report may be used to make decisions regarding a cross-correlation of wells, a true mineralogy of a section of a wellbore, and decisions that may alter a drilling operation (e.g., to keep drilling on course, to stop drilling, to alter course, to set casing points, to determine whether the drill bit is in a pay zone, etc.). Flow of the flowchart 500 ceases.

### Example Computer

FIG. 6 is a block diagram depicting an example computer, according to some implementations. FIG. 6 depicts a computer 600 for obtaining and interpreting images of cuttings from the image capture device 210. The computer 600 includes a processor 601 (possibly including multiple processors, multiple cores, multiple nodes, and/or implementing multi-threading, etc.). The computer 600 includes memory 607. The memory 607 may be system memory or any one or more of the above already described possible realizations of machine-readable media. The computer 600 also includes a bus 603 and a network interface 605. The computer 600 may communicate via transmissions to and/or from remote devices via the network interface 605 in accordance with a network protocol corresponding to the type of network interface, whether wired or wireless and depending upon the carrying medium. In addition, a communication or transmission may involve other layers of a communication protocol and or communication protocol suites (e.g., transmission control protocol, Internet Protocol, user datagram protocol, virtual private network protocols, etc.).

The computer 600 also includes a property analyzer 611 and a controller 615 which may perform the operations described herein. For example, the property analyzer 611 may obtain images of a cuttings sample from the image capture device 210 and input the images into a learning machine. In some implementations, the property analyzer 611 may include the learning machine. The learning machine may generate a standardized set of descriptions of the cuttings sample based on the received images. The controller 615 may control the automated cuttings analysis system 200 and its various components. The property analyzer 611 and the controller 615 may be in communication. Any one of the previously described functionalities may be partially (or entirely) implemented in hardware and/or on the processor 601. For example, the functionality may be implemented with an application specific integrated circuit, in logic implemented in the processor 601, in a co-processor on a peripheral device or card, etc. Further, realizations may include fewer or additional components not illustrated in FIG. 6 (e.g., video cards, audio cards, additional network interfaces, peripheral devices, etc.). The processor 601 and the network interface 605 are coupled to the bus 603. Although illustrated as being coupled to the bus 603, the memory 607 may be coupled to the processor 601.

While the aspects of the disclosure are described with reference to various implementations and exploitations, it will be understood that these aspects are illustrative and that the scope of the claims is not limited to them. In general, techniques for automated cuttings analyses as described herein may be implemented with facilities consistent with any hardware system or hardware systems. Many variations, modifications, additions, and improvements are possible.

Plural instances may be provided for components, operations or structures described herein as a single instance. Finally, boundaries between various components, operations and data stores are somewhat arbitrary, and particular operations are illustrated in the context of specific illustrative configurations. Other allocations of functionality are envisioned and may fall within the scope of the disclosure. In general, structures and functionality presented as separate components in the example configurations may be implemented as a combined structure or component. Similarly, structures and functionality presented as a single component may be implemented as separate components. These and other variations, modifications, additions, and improvements may fall within the scope of the disclosure.

### Example Implementations

Implementation #1: A method for analyzing cuttings from a plurality of depths while drilling a wellbore in a subsurface formation, the method comprising: obtaining cuttings samples from the plurality of depths while drilling the wellbore in the subsurface formation; performing the following operations for each of the cuttings samples, loading a cuttings sample into a viewing area of a microscope coupled to an image capture device and a computer having a learning machine, performing analyses on the cuttings sample, and capturing, via the image capture device, a plurality of images of the cuttings sample through the microscope; and outputting a standardized cuttings report generated by the learning machine.

Implementation #2: The method of claim 1, wherein performing the analyses further comprises: illuminating the cuttings sample via one or more light sources at a plurality of light spectra; and dosing, via one or more autodosers coupled to one or more fluid storage vessels, each cuttings sample with one or more chemicals.

Implementation #3: The method of claim 1, wherein loading the cuttings sample into the viewing area comprises loading an autoloader configured to move the cuttings sample into the viewing area of the microscope.

Implementation #4: The method of claim 1, further comprising: determining, via the learning machine, one more standardized cuttings descriptors based, at least in part, on the performed analyses.

Implementation #5: The method of claim 3, further comprising: loading each cuttings sample into a cartridge configured for placement into the autoloader, wherein the autoloader is configured to move the cartridge into the viewing area of the microscope.

Implementation #6: The method of claim 1, further comprising: associating, via the learning machine, each image of the plurality of images to a depth in the wellbore; determining, via the learning machine, one or more properties of each of the cuttings samples based, at least in part, on the plurality of images; and determining, via the learning machine, a mineralogy at one or more depths in the wellbore based, at least in part, on the plurality of images and the one or more properties of each of the cuttings samples.

Implementation #7: The method of claim 6, further comprising: generating the standardized cuttings report based, at least in part, on the one or more properties of each of the cuttings and the mineralogy at the one or more depths; and performing a subsurface operation based on the standardized cuttings report.

Implementation #8: An automated cuttings analysis system for analyzing cuttings from a plurality of depths while drilling a wellbore in a subsurface formation, the automated cuttings analysis system comprising: a microscope coupled to an image capture device; an autoloader configured to place a cuttings sample obtained from the wellbore within a viewing area of the microscope; a processor; and a computer-readable medium having instructions executable by the processor, the instructions including: instructions to move, via the autoloader, the cuttings sample into the viewing area, instructions to perform one or more analyses on the cuttings sample, and instructions to generate, via a learning machine, a standardized cuttings report based, at least in part, on the analyses.

Implementation #9: The automated cuttings analysis system of claim 8, further comprising: one or more light sources, wherein the instructions to perform the one or more analyses comprise instructions to illuminate the cuttings sample in one or more light spectra.

Implementation #10: The automated cuttings analysis system of claim 8, further comprising: one or more fluid storage vessels; and one or more autodosers coupled to the one or more fluid storage vessels, wherein the instructions to perform the one or more analyses comprise instructions to dose the cuttings sample with a chemical output from the one or more autodosers.

Implementation #11: The automated cuttings analysis system of claim 8, wherein the image capture device is a CCD camera.

Implementation #12: The automated cuttings analysis system of claim 8, wherein the instructions further comprise: instructions to obtain a plurality of images of the cuttings sample via the image capture device; instructions to associate, via the learning machine, each image of the plurality of images to a depth in the wellbore; instructions to determine, via the learning machine, one or more properties of each of the cuttings sample based, at least in part, on the plurality of images; and instructions to determine, via the learning machine, a mineralogy at one or more depths in the wellbore based, at least in part, on the plurality of images and the one or more properties of each of the cuttings samples.

Implementation #13: The automated cuttings analysis system of claim 12, wherein the instructions further comprise: instructions to generate the standardized cuttings report based, at least in part, on the one or more properties of each of the cuttings and the mineralogy at the one or more depths; and instructions to perform a subsurface operation based on the standardized cuttings report.

Implementation #14: One or more non-transitory machine-readable media including instructions executable by a processor to cause the processor to analyze cuttings from a plurality of depths while drilling a wellbore in a subsurface formation, the instructions comprising: instructions to load a cuttings sample obtained from the wellbore into a viewing area of a microscope coupled to an image capture device and a computer, the computer having a learning machine; instructions to perform one or more analyses on the cuttings sample, and instructions to capture, via the image capture device, a plurality of images of the cuttings sample through the microscope; and instructions to output, via the learning machine, a standardized cuttings report based on the plurality of images.

Implementation #15: The machine-readable media of claim 14, wherein the instructions to perform the analyses comprise instructions to: illuminate the cuttings sample via one or more light sources at a plurality of light spectra; and dose, via one or more autodosers coupled to one or more fluid storage vessels, each cuttings sample with one or more chemicals.

Implementation #16: The machine-readable media of claim 14, wherein the instructions to load the cuttings sample into the viewing area comprise instructions to load an autoloader configured to move the cuttings sample into the viewing area of the microscope.

Implementation #17: The machine-readable media of claim 15, further comprising instructions to: determine, via the learning machine, one more standardized cuttings descriptors based, at least in part, on the performed one or more analyses.

Implementation #18: The machine-readable media of claim 16, further comprising instructions to: load each cuttings sample into a cartridge configured for placement into the autoloader, wherein the autoloader is configured to move the cartridge into the viewing area of the microscope.

Implementation #19: The machine-readable media of claim 14, further comprising instructions to: associate, via the learning machine, each image of the plurality of images to a depth in the wellbore; determine, via the learning machine, one or more properties of each of the cuttings sample based, at least in part, on the plurality of images; and determine, via the learning machine, a mineralogy at one or more depths in the wellbore based, at least in part, on the plurality of images and the one or more properties of each of the cuttings samples.

Implementation #20: The machine-readable media of claim 19, further comprising instructions to: generate the standardized cuttings report based, at least in part, on the one or more properties of each of the cuttings and the mineralogy at the one or more depths; and perform a subsurface operation based on the standardized cuttings report.

Use of the phrase “at least one of” preceding a list with the conjunction “and” should not be treated as an exclusive list and should not be construed as a list of categories with one item from each category, unless specifically stated otherwise. A clause that recites “at least one of A, B, and C” may be infringed with only one of the listed items, multiple of the listed items, and one or more of the items in the list and another item not listed.

As used herein, the term “or” is inclusive unless otherwise explicitly noted. Thus, the phrase “at least one of A, B, or C” is satisfied by any element from the set {A, B, C} or any combination thereof, including multiples of any element.

1. A method for analyzing cuttings from a plurality of depths while drilling a wellbore in a subsurface formation, the method comprising:
obtaining cuttings samples from the plurality of depths while drilling the wellbore in the subsurface formation;
performing the following operations for each of the cuttings samples,
loading a cuttings sample into a viewing area of a microscope coupled to an image capture device and a computer having a learning machine,
performing analyses on the cuttings sample, and
capturing, via the image capture device, a plurality of images of the cuttings sample through the microscope; and
outputting a standardized cuttings report generated by the learning machine.

2. The method of claim 1, wherein performing the analyses further comprises:
illuminating the cuttings sample via one or more light sources at a plurality of light spectra; and
dosing, via one or more autodosers coupled to one or more fluid storage vessels, each cuttings sample with one or more chemicals.

3. The method of claim 1, wherein loading the cuttings sample into the viewing area comprises loading an autoloader configured to move the cuttings sample into the viewing area of the microscope.

4. The method of claim 1, further comprising:
determining, via the learning machine, one more standardized cuttings descriptors based, at least in part, on the performed analyses.

5. The method of claim 3, further comprising:
loading each cuttings sample into a cartridge configured for placement into the autoloader, wherein the autoloader is configured to move the cartridge into the viewing area of the microscope.

6. The method of claim 1, further comprising:
associating, via the learning machine, each image of the plurality of images to a depth in the wellbore;
determining, via the learning machine, one or more properties of each of the cuttings samples based, at least in part, on the plurality of images; and
determining, via the learning machine, a mineralogy at one or more depths in the wellbore based, at least in part, on the plurality of images and the one or more properties of each of the cuttings samples.

7. The method of claim 6, further comprising:
generating the standardized cuttings report based, at least in part, on the one or more properties of each of the cuttings and the mineralogy at the one or more depths; and
performing a subsurface operation based on the standardized cuttings report.

8. An automated cuttings analysis system for analyzing cuttings from a plurality of depths while drilling a wellbore in a subsurface formation, the automated cuttings analysis system comprising:
a microscope coupled to an image capture device;
an autoloader configured to place a cuttings sample obtained from the wellbore within a viewing area of the microscope;
a processor; and
a computer-readable medium having instructions executable by the processor, the instructions including:
instructions to move, via the autoloader, the cuttings sample into the viewing area,
instructions to perform one or more analyses on the cuttings sample, and
instructions to generate, via a learning machine, a standardized cuttings report based, at least in part, on the analyses.

9. The automated cuttings analysis system of claim 8, further comprising:
one or more light sources, wherein the instructions to perform the one or more analyses comprise instructions to illuminate the cuttings sample in one or more light spectra.

10. The automated cuttings analysis system of claim 8, further comprising:
one or more fluid storage vessels; and
one or more autodosers coupled to the one or more fluid storage vessels, wherein the instructions to perform the one or more analyses comprise instructions to dose the cuttings sample with a chemical output from the one or more autodosers.

11. The automated cuttings analysis system of claim 8, wherein the image capture device is a CCD camera.

12. The automated cuttings analysis system of claim 8, wherein the instructions further comprise:
instructions to obtain a plurality of images of the cuttings sample via the image capture device;
instructions to associate, via the learning machine, each image of the plurality of images to a depth in the wellbore;
instructions to determine, via the learning machine, one or more properties of each of the cuttings sample based, at least in part, on the plurality of images; and
instructions to determine, via the learning machine, a mineralogy at one or more depths in the wellbore based, at least in part, on the plurality of images and the one or more properties of each of the cuttings samples.

13. The automated cuttings analysis system of claim 12, wherein the instructions further comprise:
instructions to generate the standardized cuttings report based, at least in part, on the one or more properties of each of the cuttings and the mineralogy at the one or more depths; and
instructions to perform a subsurface operation based on the standardized cuttings report.

14. One or more non-transitory machine-readable media including instructions executable by a processor to cause the processor to analyze cuttings from a plurality of depths while drilling a wellbore in a subsurface formation, the instructions comprising:
instructions to load a cuttings sample obtained from the wellbore into a viewing area of a microscope coupled to an image capture device and a computer, the computer having a learning machine;
instructions to perform one or more analyses on the cuttings sample, and
instructions to capture, via the image capture device, a plurality of images of the cuttings sample through the microscope; and
instructions to output, via the learning machine, a standardized cuttings report based on the plurality of images.

15. The machine-readable media of claim 14, wherein the instructions to perform the analyses comprise instructions to:
illuminate the cuttings sample via one or more light sources at a plurality of light spectra; and
dose, via one or more autodosers coupled to one or more fluid storage vessels, each cuttings sample with one or more chemicals.

16. The machine-readable media of claim 14, wherein the instructions to load the cuttings sample into the viewing area comprise instructions to load an autoloader configured to move the cuttings sample into the viewing area of the microscope.

17. The machine-readable media of claim 14, further comprising instructions to:
determine, via the learning machine, one more standardized cuttings descriptors based, at least in part, on the performed one or more analyses.

18. The machine-readable media of claim 16, further comprising instructions to:
load each cuttings sample into a cartridge configured for placement into the autoloader, wherein the autoloader is configured to move the cartridge into the viewing area of the microscope.

19. The machine-readable media of claim 14, further comprising instructions to:
associate, via the learning machine, each image of the plurality of images to a depth in the wellbore;
determine, via the learning machine, one or more properties of each of the cuttings sample based, at least in part, on the plurality of images; and
determine, via the learning machine, a mineralogy at one or more depths in the wellbore based, at least in part, on the plurality of images and the one or more properties of each of the cuttings samples.

20. The machine-readable media of claim 19, further comprising instructions to:
generate the standardized cuttings report based, at least in part, on the one or more properties of each of the cuttings and the mineralogy at the one or more depths; and
perform a subsurface operation based on the standardized cuttings report.
